(19) United States
(12) Patent Application Publication
(10) Pub. No.: US 2022/0040368 A1
(43) Pub. Date: Feb. 10, 2022

(54) ODOR/TOXIN-ABSORBING SANITARY PADS, PANTY LINERS, AND TAMPONS

(71) Applicant: Quadesha Halevi, Charlotte, NC (US)

(72) Inventor: Quadesha Halevi, Charlotte, NC (US)

(21) Appl. No.: 17/444,517

(22) Filed: Aug. 5, 2021

Related U.S. Application Data
(60) Provisional application No. 62/706,208, filed on Aug. 5, 2020.

(51) Int. Cl. A61L 15/46; A61F 13/20; A61F 13/472; A61F 13/511; A61F 13/514; A61F 13/56; A61F 13/84; A61L 15/22; A61L 15/26; A61L 15/18

(52) U.S. Cl. CPC A61L 15/46; A61F 13/2071; A61F 13/2074; A61F 13/472; A61F 13/51113; A61F 2013/4708; A61F 13/5611; A61F 13/8405; A61L 15/225; A61L 15/26; A61L 15/18; A61F 13/51405

(57) ABSTRACT

An odor and menses absorption device includes a user contact layer and at least one adjoining super absorbent core layer. Fluid can move from the user contact layer to the super absorbent core layer. The user contact layer contains a blend of polylactic acid fiber and bamboo charcoal fiber and acts as a barrier to fluids with a surface tension greater than 30 dynes.

### CROSS-REFERENCE TO RELATED APPLICATION

This application claims the benefit of priority of U.S. provisional application No. 62/706,208, filed Aug. 5, 2020, the contents of which are herein incorporated by reference.

### BACKGROUND OF THE INVENTION

The present invention relates to sanitary pads, panty liners, and tampons and, more particularly, to odor- and toxin-absorbing sanitary pads.

Sanitary menstrual pads/napkins are worn to absorb menstrual discharge while protecting clothing and furnishings. Commercially available sanitary pads often contain harmful synthetic chemicals which cause health issues. Moreover, a woman using commercially available sanitary pads must endure offensive odors, from the menses alone or in combination with unpleasant fragrances that do nothing to mask the odor.

As can be seen, there is a need for a sanitary pad that absorbs unpleasant odors while removing toxins (unwanted substances) and is made of naturally derived materials.

The present invention provides a sanitary pad that removes toxins and the peculiar smell of female menstruation without any harmful chemicals, additives, or dyes.

### SUMMARY OF THE INVENTION

In one aspect of the present invention, an odor and menses absorption device is provided, comprising a user contact layer containing a blend of polylactic acid fiber and bamboo charcoal fiber and at least one super absorbent core layer adjoining the user contact layer and in fluid communication therewith; wherein the user contact layer is operative as a barrier to fluids with a surface tension greater than 30 dynes.

These and other features, aspects and advantages of the present invention will become better understood with reference to the following drawings, description, and claims.

### BRIEF DESCRIPTION OF THE DRAWINGS

FIG. 1 is a perspective view of a sanitary napkin according to an embodiment of the present invention, shown in use;

FIG. 2 is a top plan view thereof;

FIG. 3 is a bottom plan view thereof;

FIG. 4 is a side elevation view thereof;

FIG. 5 is a semi-exploded perspective view thereof;

FIG. 6 is a cross-sectional view thereof; and

FIG. 7 is a detail cross-sectional view indicated by line 7-7 of FIG. 6.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a sanitary pad, pantyliner, and/or tampon with a top layer comprising a blend of polylactic acid fiber and bamboo charcoal fiber, which serves as an odor and menses absorption device.

The inventive pad comprises a series of layers. This layer construction, when stretched, allows breathability for moisture vapor while maintaining a barrier for fluids with surface tensions greater than 30 dynes, such as water, blood, and other bodily fluids.

The top layer, i.e., the layer used against the user's skin, may comprise a blend of polylactic acid (PLA) fiber and bamboo charcoal fiber. Bamboo charcoal absorbs odor and toxins and is believed to have antimicrobial functions.

In some embodiments, the polylactic acid fiber/bamboo charcoal fiber blend further comprises milk silk, i.e., casein fiber or a blend thereof, for comfort.

The invention generally comprises a multilayer core of super absorbent polymers (SAP) to fully absorb and lock in fluids. A suitable absorbent material may be, for example, AQUA KEEP®, a sodium polyacrylate-based SAP available from SUMITOMO SEIKA CHEMICALS CO., LTD.

A base or bottom layer may be provided of breathable polyethylene (PE), i.e., the polyethylene base layer is air-permeable and water vapor-permeable.

In some embodiments an adhesive layer may be provided to attach an external surface of the base layer of the sanitary pad to the user's undergarments. The adhesive layer may be adhered to the external surface of the base layer. A suitable adhesive may be, for example, TECHNOMELT® available from HENKEL® KGaA.

In some embodiments, a layer of release paper (i.e., a release layer) may be provided to protect the adhesive surface prior to use. The release paper may be removably attached to the adhesive layer.

In some embodiments, the component layers may be rearranged.

In some embodiments, the pads may be individually wrapped so they are easier and more discreet to carry in a purse or bag. This wrapper may be used to wrap the soiled pads before disposing of them in appropriate receptacles.

The size of sanitary pad, panty liner, or tampon is not particularly limited.

In some embodiments, the article is configured as a sanitary napkin. In other embodiments, the article may be configured as a tampon or pantyliner comprising the same natural materials, i.e., PLA and bamboo charcoal fiber. Fewer layers may be used in these embodiments. For example, a tampon generally does not comprise an adhesive layer or a release paper.

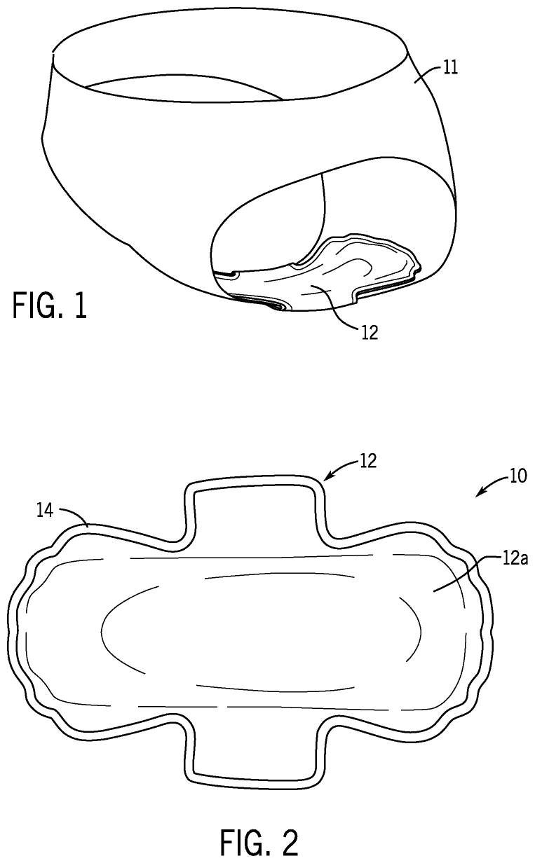

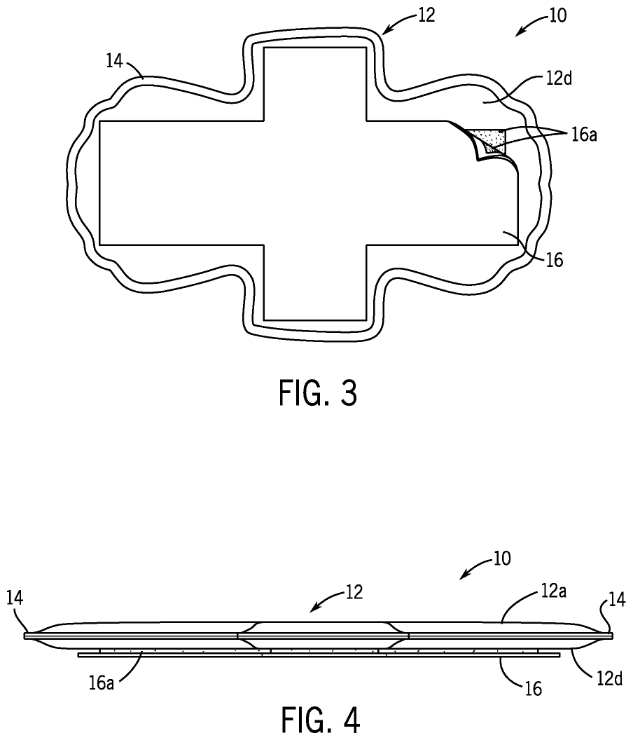

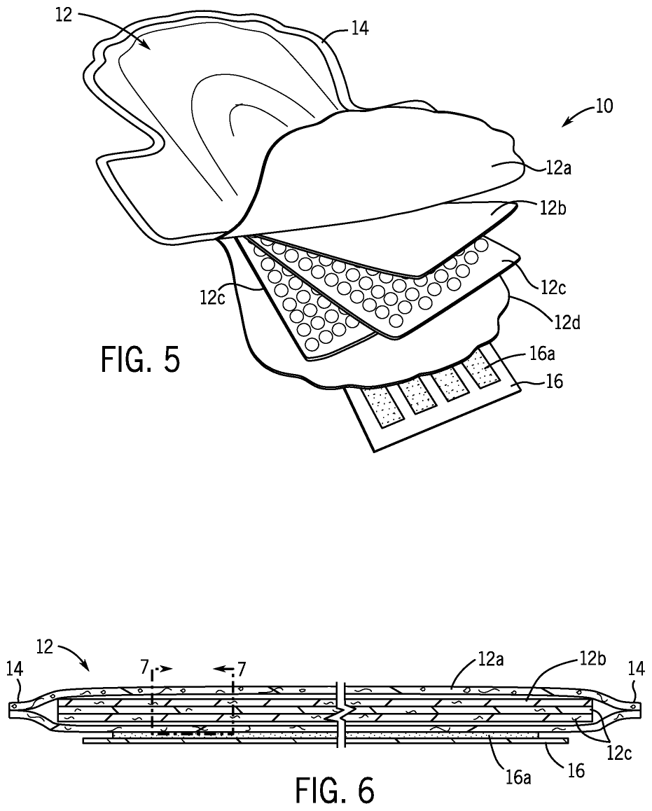

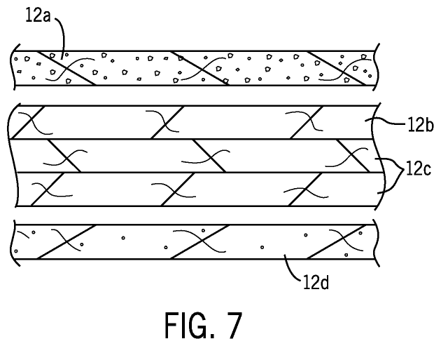

Referring to FIGS. 1 through 7, FIG. 1 shows a sanitary napkin 12 according to an embodiment of the present invention, comprising natural bamboo charcoal and casein fiber, installed on an undergarment 11. As best shown in FIGS. 2 and 3, the sanitary napkin 12 may have a pair of scalloped ends and lateral flaps for better coverage. FIGS. 5 through 7 illustrate a sanitary napkin assembly 10 according to an embodiment of the present invention comprising absorbent layers 12b, 12c sealed between a polylactic acid fiber and bamboo charcoal fiber top sheet 12a and a breathable bottom layer 12d by way of an edge seal 14. The absorbent layers 12b, 12c comprise absorbent beads on sheets of substrate in a sandwich structure, as shown in FIG. 5. FIG. 2 illustrates quilting apparent on the top sheet which may prevent the absorbent layers 12b, 12c from shifting. The breathable bottom layer 12d has an adhesive 16a for adhering the napkin 10 to the undergarment 11 and a protective backing 16 to keep the adhesive 16a from adhering prior to use, as shown in FIGS. 3 through 6. FIG. 3 illustrates that the protective backing 16 and the adhesive 16a may have a cross-shape.

It should be understood, of course, that the foregoing relates to exemplary embodiments of the invention and that modifications may be made without departing from the spirit and scope of the invention as set forth in the following claims.

What is claimed is:

1. An odor and menses absorption device comprising:
a user contact layer containing a blend of polylactic acid fiber and bamboo charcoal fiber; and
at least one super absorbent core layer adjoining the user contact layer and in fluid communication therewith;
wherein the user contact layer is operative as a barrier to fluids with a surface tension greater than 30 dynes.

2. The odor and menses absorption device of claim 1, wherein the user contact layer further comprises casein fiber.

3. The odor and menses absorption device of claim 1, configured as a tampon.

4. The odor and menses absorption device of claim 1, further comprising:
a polyethylene base layer,
wherein the polyethylene base layer is air-permeable and water vapor-permeable, and
wherein the at least one super absorbent core layer is sealed between the user contact layer and the polyethylene base layer.

5. The odor and menses absorption device of claim 4, further comprising:
an adhesive layer adhered to an external surface of the polyethylene base layer; and
a release layer removably attached to the adhesive layer.

6. The odor and menses absorption device of claim 5, further comprising a pair of scalloped ends.

7. The odor and menses absorption device of claim 5, configured as a panty liner.

8. The odor and menses absorption device of claim 5, configured as a sanitary napkin.

9. The odor and menses absorption device of claim 8, further comprising lateral flaps.
